(19) United States
(12) Patent Application Publication
(10) Pub. No.: US 2007/0082044 A1
(43) Pub. Date: Apr. 12, 2007

(54) Synergistic effect of compositions comprising carotenoids selected from lutein, beta-carotene and lycopene

(75) Inventor: Kyung-Jin Yeum, Winchester, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: TRUSTEES OF TUFTS COLLEGE, Medford, MA (US)

(21) Appl. No.: 10/561,413

(22) PCT Filed: Mar. 10, 2005
(86) PCT No.: PCT/US05/07651

§ 371 Date: Nov. 24, 2006

Related U.S. Application Data
(60) Provisional application No. 60/551,742, filed on Mar. 10, 2004.

(51) Int. Cl. A61K 31/015; A61K 9/48; A61K 9/64

(52) U.S. Cl. 424/451; 424/456; 514/763

(57) ABSTRACT

The methods of the invention can be used to protect against lymphocyte DNA damage and free-radical associated disorders in a subject. The methods of the present invention can be used to increase the antioxidant capacity in both the aqueous and lipid compartments, decrease DNA oxidation, decrease lipid peroxidation, and increase antioxidant nutrient levels in the circulation. The protective effect of the physiologic dose of the mixed carotenoid supplement is rapid, consistent and cumulative.

### BACKGROUND OF THE INVENTION

Oxidative stress has been implicated in the pathogenesis of chronic diseases related to aging, such as cancer and cardiovascular disease (Benzié et al Eur J Nutr 2000; 39: 53-61). Numerous epidemiological studies have indicated that diets rich in fruits and vegetables are correlated with a reduced risk of such diseases (Liu et al Int J Epidemiol 2001; 30:130-135, Greenberg et al. JAMA 1996; 275:699-703; Gaziano & Hennekens Ann NY Acad Sci 1993; 691:148-55; Riemersma et al Lancet 1991; 337: 1-5). It is believed that the antioxidants present in the fruits and vegetables can prevent damage from harmful reactive oxygen species, which are continuously produced in the body during normal cellular functioning. Thus, a diet supplemented with antioxidants can be a part of a defense strategy to minimize oxidative damage in a vulnerable population such as the elderly.

Carotenoids, naturally-occurring pigments which are synthesized by plants, algae, bacteria, and certain animals, such as birds and shellfish have antioxidant activities. Carotenoids are a group of hydrocarbons (e.g., carotenes) and their oxygenated, alcoholic derivatives (e.g., xanthophylls), and include, for example, actinioerythrol, astaxanthin, bixin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotene, β-cryptoxanthin, lutein, lycopene, violerythrin, zeaxanthin, and esters of hydroxyl- or carboxyl-containing members thereof. As a result of a high intake of fruits and vegetables, 34 carotenoids and their metabolites are found in human serum and tissues at varying concentrations. Alpha-carotene, β-carotene, lycopene, lutein, β-cryptoxanthin, and zeaxanthin are the predominant carotenoids found in plasma.

While the in vitro protective effect of carotenoids against oxidants has been shown in recent years, their effect in vivo has not been proven. The metabolism and function of carotenoids in humans differ from that shown in in vitro studies as antioxidant nutrients can interact with each other during gastrointestinal absorption and metabolism. Most intervention trials using carotenoid supplements did not show protective effects against cancer or cardiovascular disease. For example, recent clinical studies link high beta-carotene consumption with harmful effects, including a higher incidence of lung cancer in individuals exposed to extraordinary oxidative stress (Wemer Siems et al. FASEB J. 2002 Aug; 16(10):1289-91.) In addition, results from intervention trials indicate that supplemental beta-carotene increases lung cancer incidence and mortality among smokers (Palozza P et al. Mol Aspects Med. 2003 Dec; 24(6):353-62).

Accordingly, a need exists for methods of antioxidant supplementation that can rapidly, consistently and effectively protect against DNA damage. In addition, a need exists for a combination of low levels of antioxidants that produce a protective effective effect in vivo without harmful side effects.

### SUMMARY OF THE INVENTION

The invention is based, in part, on the discovery of the synergistic effect of lutein, beta-carotene, and lycopene in decreasing oxidative damage in human lymphocytes. Methods of decreasing DNA damage through the administration of a carotenoid supplement to a subject are disclosed. Furthermore, the methods of the invention can be used to protect against certain disorders that arise from oxidative stress and the presence of excess free radicals in a subject.

Accordingly, in one aspect, the invention pertains to a method of decreasing DNA damage through the administration of a combination of carotenoids. The combination of physiological doses of lutein, β-carotene and lycopene has a synergistic effect resulting in a decrease of DNA damage that exceeds that of carotenoids given alone.

In another aspect, the combination of physiological doses of lutein, β-carotene and lycopene changes the antioxidant capacity in the aqueous and lipid compartments of plasma. In yet another aspect, the combination of lutein, β-carotene and lycopene improves DNA response to an oxidative stress. The Examples show that DNA is less susceptible to oxidative damage following supplementation of the mixture of lutein, with at least one of β-carotene and/or lycopene.

In some embodiments, the method can be practiced using a carotenoid-containing dry powder in the form of a multicore structure in which at least two cores of a multicore structure comprise one or more different carotenoids selected from the group consisting of substantially purified lutein, beta-carotene, and lycopene. In some embodiments, the invention comprises administering a carotenoid-containing dry powder in different forms, such as drink preparations, tablets, sugar coated tablets and hard and soft gelatin or cellulose capsules.

In some embodiments, the combination of carotenoids are given in a single dose. The single dose may be solid, liquid, applied topically or intravenous. In a preferred embodiment, the carotenoids are contained in a solid preparation that can be taken orally (see, for example, U.S. patent application Ser. No. 09/929,075).

A pharmaceutical composition for use in decreasing DNA damage and/or for use in protecting against a free radical associated disorder comprising an effective daily dose of about 0.1 to 20 mg lutein, and at least one of the group consisting of beta-carotene and lycopene in an amount sufficient to act synergistically with lutein, is also disclosed. The composition can further comprise at least one of about 0.1 mg to 20 mg beta-carotene or about 0.1 to 20 mg lycopene, or about 0.5 mg to 10 mg beta-carotene or about 0.5 to 10 mg lycopene. The composition can further comprises a carotenoid-containing dry powder in the form of a multicore structure in which at least two cores of a multicore structure comprise one or more different carotenoids of the group consisting of substantially purified lutein, beta-carotene, and lycopene. The carotenoid-containing dry powder can be made into different forms, including, but not limited to, drink preparations, tablets, sugar coated tablets, hard gelatin capsules and soft gelatin capsules.

In some embodiments, the solid preparation may be combined with a lipophilic component. The combination of carotenoids can also be taken in combination with dietary fat. The solid preparation may, for example, use a permissible oil, such as sesame seed oil, corn oil, cotton seed oil, soybean oil or peanut oil, and esters of medium-chain plant fatty acids at a concentration of from 0 to 500% by weight, preferably from 10 to 300% by weight, particularly preferably from 20 to 100% by weight, based on the active compounds. The solid preparation may also be taken with a meal containing a sufficient fat content (e.g. greater than 1 gram, preferably greater than 10 g, more preferably greater than 25 g) so that the substantially water immiscible carotenoids can be fully absorbed by the subject. Combining the carotenoid preparation with a lipophilic component can increase the antioxidant capacity in the aqueous and lipid compartments of plasma.

The present invention also provides a method of slowing the effects of aging by administering a synergistic combination of carotenoids to the subject, wherein the synergistic combination comprises at least two of the group consisting of lutein, beta-carotene, and lycopene. Basal DNA damage, as well as hydrogen peroxide induced DNA damage, are associated with age. Increased frequencies of micronuclei and chromosome aberrations with age suggest an increase of genetic instability with age. The present composition can reduce DNA damage, thereby slowing the effects of the aging process.

### BRIEF DESCRIPTION OF THE FIGURES

FIG. 1 is a visual classification of DNA damage according to the relative proportion of DNA in comet tail;

FIG. 2 is a bar graph showing changes in plasma total carotenoid concentrations (lutein, β-carotene and lycopene) at various times during carotenoid supplementation in women (50-70 yr);

FIG. 3 is a bar graph showing changes over time in plasma lutein concentrations in women (50-70 yr) taking carotenoid supplements;

FIG. 4 is a bar graph showing changes over time in plasma β-carotene concentrations in women (50-70 yr) taking carotenoid supplements;

FIG. 5 is a bar graph showing changes over time in plasma lycopene concentrations in women (50-70 yr) taking carotenoid supplements; and

FIG. 6 is a graph of the effect of carotenoid supplementation on basal DNA damage in women (50-70 yr).

### DETAILED DESCRIPTION OF THE INVENTION

The methods of the invention can be used to protect against lymphocyte DNA damage and free-radical associated disorders in a subject. The methods of the present invention can be used to increase the antioxidant capacity in both the aqueous and lipid compartments, decrease DNA oxidation, increase gene expression of a panel of genes affected by carotenoids, decrease lipid peroxidation, and/or increase antioxidant nutrient levels in the circulation. The protective effect of a mixed carotenoid supplement, according to the invention, is rapid, consistent and cumulative.

So that the invention is more clearly understood, the following terms are defined:

The term “free radical” as used herein refers to molecules containing at least one unpaired electron. Most molecules contain even numbers of electrons, and their covalent bonds normally consist of shared electron pairs. Cleavage of such bonds produces two separate free radicals, each with an unpaired electron (in addition to any paired electrons). They may be electrically charged or neutral and are highly reactive and usually short-lived. They combine with one another or with atoms that have unpaired electrons. In reactions with intact molecules, they abstract a part to complete their own electronic structure, generating new radicals, which go on to react with other molecules. Such chain reactions are particularly important in decomposition of substances at high temperatures and in polymerization. In the body, oxidized free radicals can damage tissues. Antioxidant may reduce these effects. Heat, ultraviolet light, and ionizing radiation all generate free radicals. Free radicals are generated as a secondary effect of oxidative metabolism. An excess of free radicals can overwhelm the natural protective enzymes such as superoxide dismutase, catalase, and peroxidase. Free radicals such as hydrogen peroxide (H₂O₂), hydroxyl radical (HO•), singlet oxygen (¹O₂), superoxide anion radical (O•₂⁻), nitric oxide radical (NO•), peroxyl radical (ROO•), peroxynitrite (ONOO⁻) can be in either the lipid or compartments.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The phrase “free radical associated disorder” as used herein refers to a pathological condition of in a subject that results at least in part from the production of or exposure to free radicals, for example, oxyradicals, or other reactive oxygen species in vivo. The term “free radical associated disorder” encompasses pathological states that are recognized in the art as being conditions wherein damage from free radicals is believed to contribute to the pathology of the disease state, or wherein administration of a free radical inhibitor (e.g., desferrioxamine), scavenger (e.g., tocopherol, glutathione), or catalyst (e.g., SOD, catalase) are shown to produce a detectable benefit by decreasing symptoms, increasing survival, or providing other detectable clinical benefits in protecting or preventing the pathological state. Examples of free radical disorders include, but are not limited to, ischemic reperfusion injury, inflammatory diseases, systemic lupus erythematosis, myocardial infarction, stroke, traumatic hemorrhage, spinal cord trauma, Crohn's disease, autoimmune diseases (e.g., rheumatoid arthritis, diabetes), cataract formation, age-related macular degeneration, Alzheimer's disease, uveitis, emphysema, gastric ulcers, oxygen toxicity, neoplasia, undesired cell apoptosis, and radiation sickness. Such diseases can include “apoptosis-related ROS” which refers to reactive oxygen species (e.g., O₂⁻) which damage critical cellular components (e.g., lipid peroxidation) in cells stimulated to undergo apoptosis, such apoptosis-related ROS may be formed in a cell in response to an apoptotic stimulus and/or produced by non-respiratory electron transport chains (i.e., other than ROS produced by oxidative phosphorylation).

The term “oxidative stress” as used herein refers to the level of damage produced by oxygen free radicals in a subject. The level of damage depends on how fast reactive oxygen species are created and then inactivated by antioxidants.

The term “deviation” or “deviate” are used interchangeably herein and refer to a change in the antioxidant activity of a sample. The change can be an increase, decrease, elevation, or depression of antioxidant activity from a known normal value. For example, an increase or decrease of antioxidant activity in the lipid compartment of a sample, the aqueous compartment of a sample, or in both the lipid and aqueous compartment of the sample.

Carotenoids have in vitro antioxidant activity at physiological oxygen tensions (Zhang & Omaye, Toxicol in Vitro 2001; 15:13-24). However, this antioxidant effect has not been conclusively demonstrated in humans (Krinsky N I. Carotenoids and oxidative stress. In Oxidative stress and aging: Advances in basic science, diagnostics, and intervention. (Gutler R G, Rodriguez H Eds.) World Scientific Publishing Co., New York (in press)). It should be noted that the metabolism of carotenoids, and possibly their functions, differ in vivo among species. Carotenoids can interact with each other during intestinal absorption, metabolism and blood clearance, and individual responses can be very different (van den Berg & van Vliet Am J Clin Nutr 1998; 68:82-89; Paetau et al Am J Clin Nutr. 1997; 66:1133-1143; Kostic et al Am J Clin Nutr 1995; 62:604-610; White et al J Am coll Nutr 1994; 13:665-671.).

The present invention describes the antioxidant activity in human blood of a combination of the major carotenoids in fruits and vegetables, such as lutein, β-carotene and lycopene. Lutein can be obtained from green leafy vegetables, β-carotene is present in yellow and orange vegetables, and lycopene is predominantly contained in tomatoes. The synergistic effect of these carotenoids result in a protective effect against free-radical associated disorders and oxidative stress. The combination of carotenoids of the present invention has been shown in the Examples to decrease DNA damage. As shown in the Examples, the methods of this invention are based on the true antioxidant potentials of dietary antioxidants, and the interactions that may take place among these nutrients.

Carotenoids incorporate into the inner, hydrophobic part of the membrane, which can increase membrane fluidity. The structural features of the carotenoids play a role in their membrane absorption and their ability to fit into the membrane bilayer. Thus, the synergistic effect between lutein, and beta-carotene and/or lycopene can be attributed to differences in polarity. Lutein and zeazanthin are polar carotenoids, while beta-carotene and lycopene are non-polar carotenoids. Lycopene, a red-pigmented carotenoid which can be found, for example, in tomatoes comprises a long chain of conjugated double bonds, which give lycopene its ability to neutralize free radicals. In particular, lycopene is a powerful neutralizer of superoxide (O₂). Beta-carotene consists of a long nonpolar chain and will therefore be located in cell membranes and lipoproteins. Lutein is a natural fat-soluble yellowish pigment the structure of which contains hydroxyl groups. Lutein's polar structure allows it to anchor to and span the membrane, which increases membrane rigidity, while non-polar beta-carotene and lycopene can cross into the membrane.

Lutein and zeaxanthin belong to the xanthophyll class of carotenoids, also known as oxycarotenoids. These can be found in corn, egg yolks and green vegetables and fruits, such as broccoli, green beans, green peas, brussel sprouts, cabbage, kale, collard greens, spinach, lettuce, kiwi and honeydew. The xanthophylls, which in addition to lutein and zeaxanthin, include alpha-and beta-cryptoxanthin, contain hydroxyl groups. This makes them more polar than carotenoids, such as beta-carotene and lycopene, which do not contain hydroxyl groups. Although lutein and zeaxanthin have identical chemical formulas and are isomers, they are not stereoisomers. They are both polyisoprenoids containing 40 carbon atoms and cyclic structures at each end of their conjugated chains. As used herein, “lutein” is intended to include lutein and all its isomers, including zeaxanthin. They both occur naturally as all-trans (all-E) geometric isomers and the principal difference between them is in the location of a double bond in one of the end rings.

The invention pertains to a method of decreasing DNA damage through the administration of a combination of carotenoids. The combination of physiological doses of lutein, β-carotene and lycopene have a synergistic effect resulting in a decrease of DNA damage that exceeds that of carotenoids given alone. The carotenoid content can range from 0.1 to 20 mg of beta-carotene, from 0.1 to 20 mg of lycopene and 0.1 to 20 mg of lutein, preferably from 0.5 to 10 mg of beta-carotene, from 0.5 to 10 mg of lycopene and from 0.5 to 10 mg of lutein, particularly preferably from 2 to 10 mg of beta-carotene, from 2 to 10 mg of lycopene and from 2 to 10 mg of lutein.

The mixture of carotenoids is given in a single dose. The single dose can be solid, liquid, applied topically or intravenous. In a preferred embodiment, the carotenoids are contained in a solid preparation that can be taken orally (see, for example, U.S. patent application Ser. No. 09/929,075). In some embodiments, the solid preparation may be combined with a lipophilic component. The utilization of carotenoids is facilitated when taken in combination with dietary fat (Ribaya-Mercado J D Nutr Rev. 2002 Apr; 60(4):104-10). The solid preparation can, for example, use a permissible oil, such as sesame seed oil, corn oil, cotton seed oil, soybean oil or peanut oil, and esters of medium-chain plant fatty acids at a concentration of from 0 to 500% by weight, preferably from 10 to 300% by weight, particularly preferably from 20 to 100% by weight, based on the active compounds. The solid preparation can also be taken with a meal containing a sufficient fat content (e.g. greater than 1 gram, preferably greater than 10 g, more preferably greater than 25 g) so that the substantially water immiscible carotenoids can be fully absorbed by the subject. Combining the carotenoid preparation with a lipophilic component increases the antioxidant capacity in the aqueous and lipid compartments of plasma.

The mixture of physiological doses of lutein, β-carotene and lycopene changes the antioxidant capacity in the aqueous and lipid compartments of plasma. The mixture of physiological doses of lutein, β-carotene and lycopene can also improve DNA response to an oxidative stress. For example, DNA is less susceptible to oxidative damage following supplementation of the mixture of physiological doses of lutein, β-carotene and lycopene. Thus, the methods of the present invention can be used to protect against a free radical associated disorder.

As shown in the Examples, DNA damage in human lymphocytes was decreased following consumption of a combination of carotenoids for 8 weeks. The Examples compare the DNA damage following consumption of individual carotenoids (12 mg of one of lutein, β-carotene or lycopene) to a combination of lutein, β-carotene and lycopene (4 mg each). The combination was shown to produce rapid DNA protection at low doses. The three carotenoids were found to have a synergistic effect. This may be due to the differences in their polarity (i.e., lutein is more polar; lycopene has more conjugation) so that when taken together their functional bioavailability is increased.

Carotenoid Supplement

Carotenoid supplements useful for the present invention can be produced using a number of methods as disclosed in the patent literature for formulating carotenoids. For example, EP-A-0 065 193 and EP-A-0 937 412 describe processes for converting carotenoids into finely divided pulverulent forms. EP-A-0498 824 discloses a process for grinding carotenoids in a protective-colloid-containing aqueous medium and subsequent conversion of this dispersion into a dry powder. EP-A-0 410 236 relates to a process for producing colloidal carotenoid preparations by contacting a suspension of a carotenoid in a high-boiling oil with superheated steam, emulsifying this mixture in an aqueous protective colloid solution and subsequent drying. WO 98/26008 describes a process for producing stable aqueous dispersions and dry powders of xanthophylls. WO 99/48487 describes preparations of carotenoid mixtures in which the carotenoids originate from natural sources. Owing to the high phospholipid content in these preparations, together with a high viscosity of the oily dispersion, the service properties of this formulation are not always satisfactory.

The abovementioned preparations, when carotenoid mixtures are used, not infrequently encounter problems with stability and bioavailability. In addition, in the case of mixtures having extremely different contents of the individual carotenoids, formation of aggregates among the carotenoids can lead to unwanted inhomogeneous distributions of the active compounds in these preparations. Furthermore, mixtures of dry powders of individual carotenoids also frequently display separation during transport or storage.

In a preferred embodiment, solid preparations of carotenoids can be used. The preferred solid preparation of active carotenoid compounds useful for the present invention is suitable for the food sector and animal feed sector or for pharmaceutical and cosmetic applications having a multicore structure, in particular carotenoid-containing dry powders, a process for their production and the use of these solid preparations for producing food supplements and as additive to foods, animal feeds, pharmaceutical and cosmetic preparations is described in U.S. patent application Ser. No. 09/929,075.

Stable, homogeneous equal distribution of active compounds can be enhanced by administering the compounds in the form of a multicore structure in which at least two cores of a multicore structure comprise one or more different carotenoids of the group consisting of substantially purified lutein, beta-carotene, and lycopene. The multicore structure is a particle species (secondary particle) having a mean particle size of from 5 to 3000 μm, preferably from 10 to 2500 μm, particularly preferably from 50 to 2000 μm, very particularly preferably from 100 to 1000 μm, in which further particle species (primary particles), called cores, are embedded in a matrix, the cores having a mean particle size, preferably, of from 0.01 to 1.0 μm, particularly preferably from 0.03 to 0.5 μm, very particularly preferably from 0.05 to 0.2 μm.

Examples of such multicore structures are found in U.S. Pat. No. 5,780,056 and in the diagrams described there and in D. Horn and E. Luddecke: “Preparation and characterization of nano-sized carotenoid hydrosols” in Fine Particle Science and Technology, 761-775 [E. Pelizzetti (Ed.), Kluwer Academic Publishers, Netherlands, 1996] and H. Auweter et al., Angew. Chem. Int. Ed. 38 (1999) 5, 2188-91.

The primary particles of the multicore structures as described in U.S. Pat. No. 5,780,056 are identical in composition, that is to say in the case of a mixture, for example of carotenoids, each core is identical with respect to type and amount of the carotenoid individual components present therein.

A feature of the preferred solid preparations in the form of a multicore structure in which at least two cores of a multicore structure comprise one or more different carotenoids of the group consisting of substantially purified lutein, beta-carotene, and lycopene is that they firstly prevent or decrease unwanted interactions between the active compounds within the multicore structure by encapsulation of the individual active compounds, and secondly they permit more flexible organization of the production of user-friendly formulations of active-compound-containing mixtures.

The preferred supplement comprises a mixture of beta-carotene, lycopene and lutein. However, the supplement can contain other active compounds suitable for the food sector and animal nutrition sector or for pharmaceutical and cosmetic applications including, but not limited to the following compounds: Fat-soluble vitamins, for example the K vitamins, vitamin A and derivatives such as vitamin A acetate, vitamin A propionate or vitamin A palmitate, vitamin D₂ and vitamin D₃ and vitamin E and derivatives. Vitamin E in this context is natural or synthetic alpha-, beta-, gamma- or delta-tocopherol, preferably natural or synthetic alpha-tocopherol, or else is tocotrienol. Vitamin E derivatives are, for example, tocopheryl C₁-C₂₀-acyl esters such as tocopheryl acetate or tocopheryl palmitate. Water-soluble vitamins, in particular ascorbic acid and its salts such as sodium ascorbate, and vitamin C derivatives such as sodium, calcium or magnesium ascorbyl 2-monophosphate or calcium ascorbyl 2-polyphosphate, calcium pantothenate, panthenol, vitamin B₁ (thiamine), as hydrochloride, nitrate or pyrophosphate, vitamin B₂ (riboflavin) and its phosphates, vitamin B₆ and salts, vitamin B₁₂, biotin, folic acid and folic acid derivatives such as tetrahydrofolic acid, 5-methyltetrahydrofolic acid, 5-formyltetrahydrofolic acid, nicotinic acid and nicotinamide. Compounds having vitamin character or coenzyme character, for example choline chloride, carnitine, gamma-butyrobetaine, lipoic acid and salts of lipoic acid, kreatine, ubiquinones, S-methylmethionine, S-adenosylmethionine. Polyunsaturated fatty acids, for example linleoic acid, linolenic acid, arachidonic acid, eicosapentaenoic acid, docosahexaenoic acid. Food pigments such as curcumin, carmine or chlorophyll. Additional carotenoids, not only carotenes but also xanthophylls, for example alpha-carotene, astaxanthin, zeaxanthin, capsanthin, capsorubin, cryptoxanthin, citranaxanthin, canthaxanthin, bixin, beta-apo-4-carotenal, beta-apo-8-carotenal and beta-apo-8-carotenic esters. Polyphenols, for example isoflavon, genistein, daidzein, epigallocatechin gallate, green tea extract and berry extract.

The carotenoids present in the cores can be of either natural or synthetic origin. For beta carotene and lycopene they generally have a purity of at least 80%, preferably greater than 90%, particularly preferably greater than 95%, very particularly preferably greater than 98%, determined by quantitative HPLC analysis. Lutein has a purity of at least 75%, preferably greater than 80%, particularly preferably greater than 85%. In the case of carotenoids from natural sources, for example lutein or lycopene, it is possible that these compositions can comprise up to 20% of other carotenoids, for example zeaxanthine as “impurities”. “Substantially pure” as used herein, is intended to mean a purity of at least 60%, preferably greater than 70%, more preferably greater than 80%, more preferably greater than 90%, particularly preferably greater than 95%, very particularly preferably greater than 98%, determined by quantitative HPLC analysis.

A dry powder of this type comprises a multicore structure of secondary particles in which at least three primary particles have a different carotenoid composition, in each case one particle species comprising only beta-carotene, the second lycopene and the third only lutein.

The content of beta-carotene, lycopene and lutein in the inventive dry powders is generally from 0.1 to 50% by weight, preferably from 1 to 35% by weight, particularly preferably from 3 to 25% by weight, very particularly preferably from 5 to 20% by weight, based on the total amount of the formulation.

In the case of the abovementioned ternary combination, the quantitative ratio of the carotenoids present in the dry powder is 1 part of beta-carotene, from 0.02 to 20 parts of lycopene and from 0.02 to 20 parts of lutein, preferably 1 part of beta-carotene, from 0.1 to 5 parts of lycopene and from 0.1 to 5 parts of lutein, particularly preferably 1 part of beta-carotene, from 0.2 to 2 parts of lycopene and from 0.1 to 2 parts of lutein, very particularly preferably 1 part of beta-carotene, from 0.3 to 1.2 parts of lycopene and from 0.1 to 1.2 parts of lutein.

In the carotenoid formulations, in particular the abovementioned ternary combination, in addition, the phosphorus content in the formulations is less than 2.0% by weight, advantageously less than 1.0% by weight, preferably less than 0.5% by weight, particularly preferably less than 0.1% by weight, very particularly preferably less than 0.02% by weight, based on the total amount of the mixture of beta-carotene, lycopene and lutein. The low phosphorus content is at the same time associated with a small amount of phospholipids, which improves the service properties of the dry powders, for example the flowability in oily dispersions particularly at low temperatures.

The carotenoid formulations can comprise, in their secondary particles, in addition to the above-described carotenoid-containing cores, other primary particles whose active compounds do not originate from the carotenoid class of substances. These are preferably vitamin-containing primary particles.

The primary particles have a core/shell structure in which the active-compound-containing core is surrounded by a protective colloid. Suitable protective colloids are either electrically charged polymers (polyelectrolytes) or neutral polymers. Typical examples are, inter alia, gelatin, such as beef gelatin, pig gelatin or fish gelatin, starch, modified starch, dextrin, plant proteins, such as soy proteins, which may be hydrolyzed, pectin, guar gum, xanthan, gum arabic, casein, caseinate or mixtures thereof. However, use may also be made of polyvinyl alcohol, polyvinylpyrrolidone, methyl cellulose, carboxymethyl cellulose, hydroxypropyl cellulose, flake shellac and alginates. For more details see R. A. Morton, Fat Soluble Vitamins, Intern. Encyclopedia of Food and Nutrition, Vol. 9, Pergamon Press 1970, pp. 128-131.

Preferred protective colloids are compounds selected from the group consisting of gelatin, such as beef gelatin, pig gelatin and fish gelatin, plant proteins, pectin, casein, caseinate, gum arabic, modified starch and shellac. Protective colloids, which are particularly preferably useful, are aqueous solutions of modified starch, pectin, casein, caseinate and/or gum arabic.

To increase the mechanical stability of the dry powder, it is expedient to add to the colloid a plasticizer, such as sugars or sugar alcohols, for example sucrose, glucose, lactose, invert sugar, sorbitol, maltose, isomalt, mannitol or glycerol, or else polymers such as polyvinyl alcohol or polyvinylpyrrolidone. Plasticizers preferably used are sucrose, isomalt, sorbitol and lactose.

The ratio of protective colloid and plasticizer to active compound is generally chosen so that a solid preparation is obtained which comprises from 0.1 to 50% by weight of at least two active compounds, from 10 to 50% by weight, preferably from 15 to 35% by weight, of a protective colloid and from 20 to 70% by weight, preferably from 30 to 60% by weight, of a plasticizer, all percentages being based on the dry matter of the formulation and the total of the percentages of the individual components being 100%.

To increased the stability of the active compounds to oxidative degradation, it can be advantageous to add from 0 to 10% by weight, preferably from 0.5 to 7.5% by weight, based on the dry matter of the formulation, of one or more stabilizers, such as alpha-tocopherol, tert-butylated hydroxytoluene, tert-butylated hydroxyanisole, ascorbic acid or ethoxyquins.

In addition, emulsifiers can be used, for example ascorbyl palmitate, polyglycerol fatty acid esters, sorbitol fatty acid esters, propylene glycol fatty acid esters or lecithin at a concentration of from 0 to 200% by weight, preferably from 5 to 150% by weight, particularly preferably from 10 to 80% by weight, based on the active compounds used.

In some circumstances it can also be advantageous to use in addition a physiologically permissible oil, for example sesame seed oil, corn oil, cotton seed oil, soybean oil or peanut oil, and esters of medium-chain plant fatty acids at a concentration of from 0 to 500% by weight, preferably from 10 to 300% by weight, particularly preferably from 20 to 100% by weight, based on the active compounds.

The matrix present in the multicore structure is generally formed from a physiologically acceptable polymeric material. Preferably it is composed of at least one of the abovementioned protective colloids, possibly in combination with the above-described formulation aids, such as plasticizers, antioxidants and/or emulsifiers. The matrix can also comprise at least one water-soluble vitamin.

The above-described solid preparations can be produced by drying an aqueous suspension comprising at least two active compounds which are suitable for the food sector and animal feed sector or for pharmaceutical and cosmetic applications in the form of nanoparticulate particles, which comprises at least two of the nanoparticulate particles having a different chemical composition. Active compounds here are the compounds already mentioned at the outset. In a preferred embodiment, the active compounds are at least two carotenoids, in which case, particularly preferably, at least two of the nanoparticulate particles comprise one or more different carotenoids.

For reasons of stability it is advantageous in this case if the active compounds are present in the form of protective-colloid-stabilized nanoparticulate particles which have a mean particle size of, preferably, from 0.01 to 1.0 μm, particularly preferably from 0.03 to 0.5 μm, very particularly preferably from 0.05 to 0.2 μm.

The active compounds, in particular the carotenoids, used to produce the inventive preparations can be used in the form of very finely ground crystals, or preferably in the form of pre-prepared dry powders. These dry powders each comprise nanoparticulate particles of the individual carotenoids and may be produced by grinding or micronizing individual active compounds. Examples of these may be found, inter alia, in EP-A-0 065 193, EP-A-0 937 412 and in WO 91/06292. By redispersing the starting formulations in aqueous solutions and converting the dispersion again into a dry powder by processes known per se, for example spray-drying or spray-cooling, with or without addition of dusting powders to avoid agglomeration, the novel inventive preparations having the multicore structures described at the outset may be obtained. Details on spray-drying or spray-cooling may be found, inter alia, in WO 91/06292.

The inventive carotenoid formulations are suitable, inter alia, as additives for food preparations, in particular drink preparations, as agent for producing pharmaceutical and cosmetic preparations and for producing food supplement preparations in the human and animal sectors. Thus, drinks may be fortified, for example, by using the inventive water-dispersible dry powders in which are present mixtures of beta-carotene, lycopene and lutein at the concentrations already mentioned above.

It is also possible to use dry powders which comprise the inventive carotenoid combinations to enrich milk products such as yogurt, flavored milk drinks or ice cream, or milk pudding powders, baking mixes and confectionery products, for example fruit gums.

The invention also relates to food supplements, animal feeds, foods and pharmaceutical and cosmetic preparations comprising the above-described preparations, in particular carotenoid formulations of mixtures of beta-carotene, lycopene and lutein. Food supplement preparations and pharmaceutical preparations which comprise the inventive dry powders include, but are not limited to, tablets, sugar-coated tablets and hard and soft gelatin capsules. Preferred food supplement preparations are tablets into which the dry powders are co-incorporated, and soft gelatin capsules in which the carotenoid-containing multicore structures are present as oily suspension in the capsules. The carotenoid content in these capsules is from 0.1 to 20 mg of beta-carotene, from 0.1 to 20 mg of lycopene and 0.1 to 20 mg of lutein, preferably from 1 to 15 mg of beta-carotene, from 1 to 15 mg of lycopene and from 1 to 10 mg of lutein, particularly preferably from 2 to 10 mg of beta-carotene, from 2 to 10 mg of lycopene and from 2 to 10 mg of lutein.

Many disorders or diseases arise due to oxidative stress and the presence of free radicals. The methods of the present invention can be used to reduce, ameliorate, prevent, and/or treat disorders associated with antioxidant levels and excess free radicals. Populations at risk can be identified through methods known in the art (See, for example, U.S. Publication No. US 2002-0182736 A1, U.S. patent application Ser. No. 10/114,181 filed Apr. 2, 2002,which describes a method that is accurate, quick, non-invasive, which can be easily adapted for high throughput usage and diagnostic procedures). At risk populations or people who wish to reduce the risk of free-radical associated disorders can benefit from the methods of the present invention. For example, disorders that can be reduced, ameliorated, prevented, and/or treated using the methods of this invention include, but are not limited to, aging at a higher than normal rate, segmental progeria disorders, Down's syndrome; heart and cardiovascular diseases such as arteriosclerosis, adriamycin cardiotoxicity, alcohol cardiomyopathy; gastrointestinal tract disorders such as inflammatory & immune injury, diabetes, pancreatitis, halogenated hydrocarbon liver injury; eye disorders such as cataractogenesis, degenerative retinal damage, macular degeneration; kidney disorders such as autoimmune nephrotic syndromes and heavy metal nephrotoxicity; skin disorders such as solar radiation, thermal injury, porphyria: nervous system disorders such as hyperbaric oxygen, Parkinson's disease, neuronal ceroid lipofuscinoses, Alzheimer's disease, muscular dystrophy and multiple sclerosis; lung disorders such as lung cancer, oxidant pollutants (O₃,NO₂), emphysema, bronchopulmonary dysphasia, asbestos carcinogenicity; red blood cell disorder such as malaria Sickle cell anemia, Fanconi's anemia and hemolytic anemia of prematurity; iron overload disorders such as idiopathic hemochromatosis, dietary iron overload and thalassemia; inflammatory-immune injury, for example, glomerulonephritis, autoimmune diseases, rheumatoid arthritis; ischemia reflow states disorders such as stroke and myocardial infarction; liver disorder such as alcohol-induced pathology and alcohol-induced iron overload injury; and other oxidative stress disorders such as AIDS, radiation-induced injuries (accidental and radiotherapy), general low-grade inflammatory disorders, organ transplantation, inflamed rheumatoid joints and arrhythmias. The method of the invention can be used for diagnosis and prevention of a free radical induced disorder, or an oxidative stress disorder.

This invention is further illustrated by the following examples, which should not be construed as limiting. The contents of all references, patents and published patent applications cited throughout this application, are incorporated herein by reference.

### EXAMPLES

A study was undertaken to evaluate the effectiveness of the composition of the present invention and its effect on the patients. Thirty-seven healthy non-smoking post-menopausal women (50˜70 yr) were randomly assigned to one of 5 groups, to take a daily dose of mixed carotenoids (β-carotene, lutein and lycopene, 4 mg each), or 12 mg of single carotenoid (β-carotene, lutein or lycopene), or placebo for 8 weeks. Plasma carotenoid concentrations were analyzed by an HPLC system with a C30 column, and lymphocyte DNA damage was determined by a single cell gel electrophoresis (comet) assay.

After 56-day intervention, all carotenoid supplemented groups showed significantly lower endogenous DNA damage than that of baseline (P<0.05), while the placebo group did not show any significant change. The earliest significantly decreased endogenous DNA damage was found in the mixed carotenoids group, at day 15 (P<0.05). As compared to day 1, the H₂O₂ induced DNA damage levels were significantly decreased after 56-day intervention in the mixed carotenoids group, β-carotene group and lycopene group (P<0.05). The results, discussed below, indicated that carotenoid supplementation could reduce DNA damage, and that a combination of carotenoids exert efficient protection against DNA damage. The oral intake of the composition can be used either therapeutically or prophylactically to improve the health of the subject and reduce DNA damage.

Subjects

Thirty-seven non-smoking post-menopausal women (50-70 yr) were enrolled in this study. All study participants were in good health as determined by a medical history questionnaire, physical examination, and normal results for clinical laboratory tests. In order to minimize the possible variability of genetic differences, white females were recruited from the general population and screened to select those with normal hematologic parameters, normal serum albumin, normal liver function, normal kidney function, absence of fat malabsorption and no drug intake which would interfere with fat absorption, metabolism or blood clotting. Subjects with a history of kidney stones, active small bowel disease or resection, atrophic gastritis, hyperlipidemia, insulin-requiring diabetes, alcoholism, pancreatic disease, or bleeding disorders were excluded from the study. Exogenous hormone users were also excluded from the study. Subjects weighing greater than 20% above or below the NHANES median standard were excluded. Moreover, subjects were non-smokers and did not take vitamin or carotenoid supplements for at least 2 months prior to the study. All of the study participants fulfilled the following eligibility criteria: 1) no history of cardiovascular, hepatic, gastrointestinal, or renal disease; 2) no alcoholism, no smoking, no exogenous hormone use; 3) no supplemental vitamin and/or carotenoids use for >6 wks before the start the study; and 4) baseline plasma carotenoid concentrations are less than 200% of the NHANES III median level. The study protocol was approved by the Institutional Review Board of Tufts-New England Medical Center and Tufts University Health Sciences, and written informed consent was obtained from each study participant.

Study Design

Two weeks before starting the study (d-14), 10 mL of fasting blood was drawn from the subject as a check for basal levels of carotenoids, cholesterol, and triglycerides. Plasma pepsinogen was measured as a check for atrophic gastritis. Also, subjects were educated by a research dietitian to exclude foods rich in carotenoids (i.e. 2 servings of fruit and vegetable/day which is the average consumption in the U.S.) for two weeks prior to starting this study, and during study—except as provided by the Metabolic Research Unit (MRU) of the Human Nutrition Research Center on Aging (HNRC) at Tufts University. Three-day dietary records and a Food Frequency Questionnaire were obtained 2 weeks prior to initiation of the study, as a check for carotenoid consumption.

Subjects (50-70 yr, n=37) were housed at the MRU for the first two days of the study. On the first day of the study, subjects were randomly assigned to take either 1) placebo, 2) 4 mg each of lutein, β-carotene and lycopene, 3) 4 mg of lutein, 4) 4 mg of β-carotene, or 5) 4 mg of lycopene with a meal containing 25 g of fat. Subjects were provided with a two-week supply of placebo or carotenoids along with instructions how to consume the supplements while being maintained with a low carotenoid diet on each sampling day (days 1, 15, 29 & 43). In particular, they were instructed to take the carotenoid supplements with their first meal of the day, and this food source should include 10 g of fat to insure maximum absorption of the carotenoid supplement. 10 mL of blood will be drawn at 0 (fasting), 2, 4, 6, 8, 10, 12 and 14 hours after the carotenoid dose to obtain information on the early kinetics of carotenoid absorption and tissue uptake. The subjects had the option to have an intravenous line inserted for blood drawing (I.V.). If they chose this option, 12 mL of blood was drawn for each sample and the first 2 mL of blood was discarded. Chylomicrons (the triglyceride-rich fraction of plasma) were isolated and analyzed for carotenoids to determine the plasma response kinetics in these 14 hr samples. Thereafter, subjects were discharged from the HNRC with a two-week supply of placebo or carotenoids along with instructions on how to consume the doses while being maintained on a low carotenoid diet.

From the second day of intervention, subjects took either 1) placebo, 2) 4 mg of lutein, 4 mg of β-carotene and 4 mg of lycopene, 3) 12 mg of lutein, 4) 12 mg of β-carotene, or 5) 12 mg of lycopene. On study days 15, 29, 43, and 57, overnight fasting bloods (10 mL) were collected, and 1) plasma carotenoids, 2) antioxidant capacity in both the aqueous and lipid compartments, 3) lipid peroxidation, and 4) DNA damage will be measured in these samples. In addition, 70 mL of fasting blood was collected at days 1 and 57 for the analysis of gene expression profiling in peripheral blood mononuclear cells using high-density filter-based cDNA microarrays. On study day 57, an additional 3 ml of blood was collected to measure the hemoglobin level.

Carotenoid supplements were provided to the volunteers on each sampling day while at the HNRC. The carotenoid supplements were supplied by BASF Corporation (Ludwigshafen, Germany). Dietary compliance was monitored by analyzing serum carotenoid concentrations, counting remaining pills, and by evaluating three-day dietary records and a Food Frequency Questionnaire bi-weekly. The research dietician at the HNRC also interviewed study participants at each sampling day.

The total amount of blood collected for the study was 273 mL or 289 mL if drawn by I.V. A total of 273 mL or 289 mL of blood was drawn during the 8 wk period of entire study. The quantity of blood drawn has no known effects on health. Also, a study physician clinically reviewed the hemoglobin level of each subject at study day 57, and if needed, subjects were supplemented with iron. During blood drawing there is a small risk of bruising, bleeding or pain at the site of venous puncture. There is no known risk in taking supplemental carotenoids in the amounts given for this study. The low carotenoid diets (i.e. 2 servings of fruit and vegetable/day which is the average consumption in the U.S.) required prior to and during the study posed no risk to the subjects.

Analytical Techniques

Blood samples were protected from light and centrifuged within 1 h for 15 min at 1000× g at 4° C., to separate plasma from red blood cells. Aliquots of plasma were stored at −70° C. until analyzed.

Plasma Carotenoid Analysis:

all-trans-β-Carotene (type IV), α-carotene, and lycopene were purchased from Sigma Chemical Co (St Louis). Lutein was purchased from Kemin Industries (Des Moines, Iowa). Zeaxanthin, cryptoxanthin, 13-cis-β-carotene, 9-cis-β-carotene, and echinenone were gifts from Hoffinann-La Roche Nutrley, N.J.). All HPLC solvents were obtained from JT Baker Chemical and were filtered through a 0.45-μm membrane filter before use.

Plasma carotenoid concentrations were measured by a HPLC system as previously described with minor modification (Yeum K J et al. Am J Clin Nutr 1996; 64:594-602). Plasma sample (200 μL) was extracted with 2 mL of chloroform:methanol (2: 1) followed by 3 mL of hexane. Samples were dried under nitrogen and resuspended in 75 μL ethanol:methyl tert-butyl ether (2:1) of which 25 μL was injected onto the HPLC. The HPLC system consisted of a Waters 2695 Separation Module, 2996 Photodiode Array Detector, a Waters 2475 Multi X Fluorescence Detector, a C30 carotenoid column (3 μm, 150×3.0 mm, YMC, Wilmington, N.C.), and a Waters Millenium 32 data station. The mobile phase was methanol:methyl tert-butyl ether:water (85:12:3 with 1.5% ammonium acetate in water; solvent A) and methanol:methyl tert-butyl ether:water (8:90:2 with 1% ammonium acetate in water; solvent A). The gradient procedure has been reported earlier (Yeum K J et al. Am J Clin Nutr 1996; 64:594-602). Results were adjusted by an internal standard containing echinenone and retinyl acetate. The CV for interassay (n=25) is 4% and intra assay Is 4% (n=9). Recovery of the internal standard averages 97%. The accuracy, determined by the recovery of added β-carotene to a plasma sample, averaged 95%.

Measurement of Antioxidant Nutrients in Plasma:

Plasma and chylomicron carotenoids were extracted using an enzyme extraction method, which gives 30-50% higher yield as compared to those of conventional extraction methods (Yeum et al Am J Clin Nutr 1996; 64:594-602), were measured by an HPLC system. Plasma concentrations of ascorbic acid (reduced form) and uric acid were determined by HPLC with an Electrochemical detector (ESA Inc., Bedford, Mass.).

Selective Measurement of Antioxidant Capacity Both in the Lipid and Aqueous Compartments:

Aqueous and lipid plasma oxidation were induced at a constant rate by the lipophilic azo-initiator, MeO-AMVN. Plasma oxidation was measured fluorimetrically using fluorescent probe, C11-BODIPY 581/591 (BODIPY) (Aldini et al Free Radic Biol Med. 2001 Nov 1; 31(9):1043-50).

Measurement of Lipid Peroxidation:

Lipid peroxidation was assessed by the measurement of malondialdehyde (MDA) using an HPLC system (Templar et al Nephrol Dial Transplant 2000; 14:946-951). Also, F₂-isoprostanes were measured using Mass Spectrometry (Morrow & Roberts Methods Enzymol 1999; 3000:3-12).

Measurement of DNA Oxidation Using Single Cell Gel Electrophoresis Analysis:

DNA breaks and oxidized pyrimidine bases were measured using the alkaline comet assay (Duthie et al Cancer Res 1996; 56:1291-1295). The comet assay, also called the Single Cell Gel Assay, was used to detect DNA damage and repair at the level of single cells. The Comet Assay is a rapid, sensitive test for DNA damage detection (e.g., single- and double-strand breaks, oxidative-induced base damage, and DNA-DNA/DNA-protein cross linking) by electrophoresis. The Comet Assay involves the following steps: 1. Slide preparation (i.e., mixing of cells with low melting agarose, and spread over glass microscope slides); 2. Lysis: (i.e., lysis of cell membrane and other proteins); 3. Unwinding of DNA; 4. Electrophoresis; 5. Neutralization; and 6. Staining and scoring. Cells embedded in agarose on a microscope slide are lysed with non-ionic detergent and high salt, leaving supercoiled matrix-attached DNA in a nucleoid. Under alkaline electrophoresis, DNA with breaks extends towards the anode, forming a “comet tail” when viewed by fluorescence microscopy. The percentage of total fluorescence in the tail is linearly related to DNA break frequency up to about 2 per 10⁹ daltons.

Lymphocyte separation: Lymphocytes were separated immediately after blood samples were collected. Lymphocytes were isolated by density gradient sedimentation (Histopaque 1077, Sigma diagnostic, St. Louis, USA) and frozen in 50% fetal calf serum, 40% culture medium (RPMI 1640, Sigma diagnostic, St. Louis, USA ) and 10% dimethyl sulfoxide to −-80° C. at −1° C./min freezing rate before store in liquid nitrogen.

Cryopreserved lymphocytes recovery: Cells were recovered by submerging in 37° C. water bath until last trace of ice has melted. Cells were transferred to prechilled 50% RPMI 1640 medium and 50% fetal calf serum and centrifuged at 200 g for 5 min at 4° C. Cells were resuspended in cold PBS and checked for viability (typically 95% viability) and cell number (typically 1×10⁵ cells/mL). The lymphocytes of five time points (d1, 15, 29, 43 & 57) were recovered at the same time.

Alkaline single cell gel electrophoresis: DNA strand breaks were measured in lymphocytes with the alkaline single cell gel electrophoresis, comet assay, (Collins A R. Methods Mol Biol 2002; 203:163-77) with minor modifications. The endogenous DNA damage as well as hydrogen peroxide challenged DNA damage were determined by exposing the agarose embedded with cells to PBS or H₂O₂ in PBS (10 μM) for 10 min respectively.

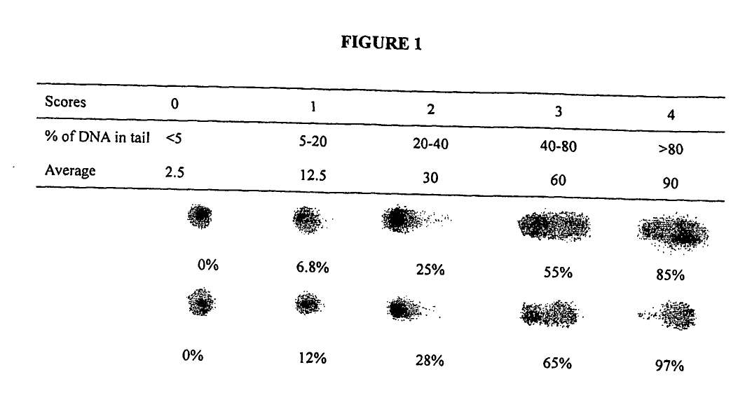

Quantitation of DNA damage: The DNA damage was determined by visual image analysis (Collins A R et al. Methods Mol Biol 2002; 186:147-59). The comets were classified visually into five categories (0-4) according to the appearance resulting from the relative proportion of DNA in tale as shown in FIG. 1. At least 100 cells were counted and categorized to avoid selection bias. Percent DNA in the tail (2.5*Cells₀+12.5*Cells₁+30*Cells₂+60*Cells₃+90*Cells₄)/Σ cells) was also calculated to express the level of DNA damage.

Statistics

At total sample size of 37 subjects was used. The sample size was based upon the plasma carotenoid response data from a study using high fruit and vegetable diet (Yeum et al Am J Clin Nutr 1996; 64:594-602), and from previous observations of plasma responses following carotenoid supplementation at doses similar to those in this study over 4 weeks. The sample size calculations were based on applying a logarithmic transformation to the data and were obtained by using the program PC-size (Dallal Am Statistician 1986; 40:52) which implements methods from Snedecor and Cochran (Statistical Methods. 6^{th} ed. The Iowa State University Press. Ames, Iowa, 1967) except that a non-central F distribution was used in the place of a non-central chi-squared distribution in order to accommodate smaller sample sizes. Results are expressed as Mean±SEM and the significance of differences were determined by Student's t test or analysis of variance using the SYSTAT 9.1 (SPSS Inc., Chicago, Ill.). If the F statistic is significant (p<0.05), the Fisher least significance test was used to determine the differences between treatments at p<0.05 unless otherwise specified. One way ANOVA was used to determine the effect of carotenoid supplementations on plasma levels and endogenous and hydrogen peroxide challenged DNA damage. Bivariate Correlation model was applied to evaluate the correlation between variables (plasma concentrations of carotenoids, tocopherols vs. DNA damage). Statistical analyses were performed using SYSTAT (version 10.2, SYSTAT Software, Inc., Point Richmond, Calif.) and SPSS (version 11.5, SPSS Inc, Chicago, Ill.).

Results

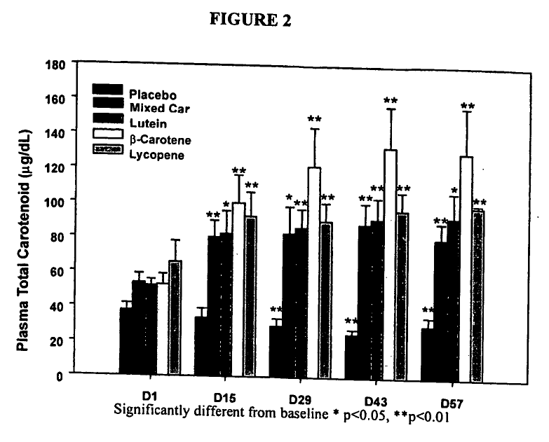

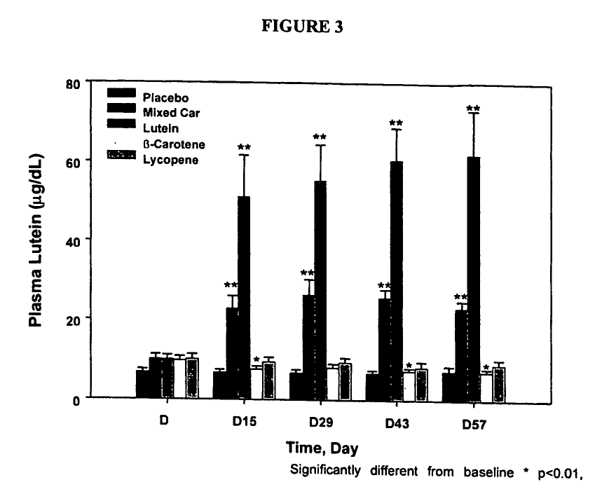

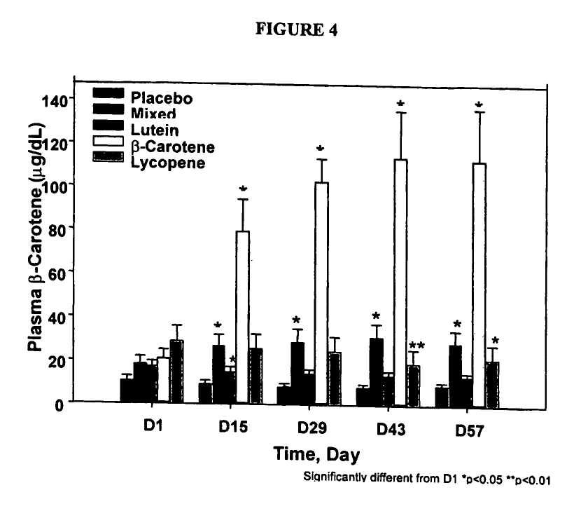

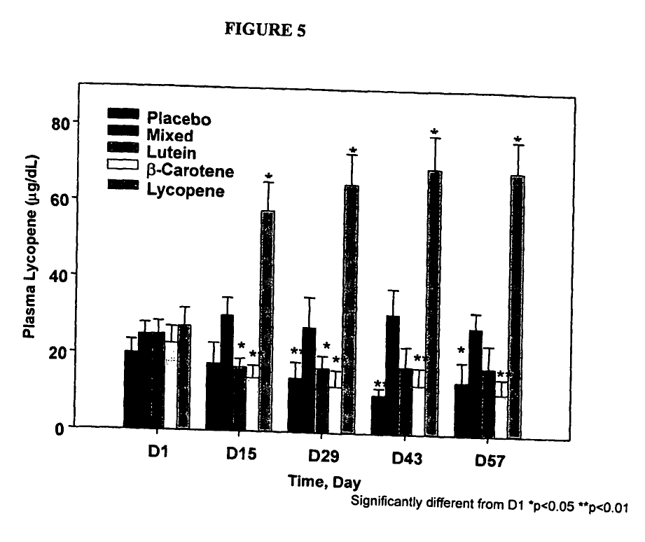

The mean±SEM baseline concentrations of the measurable plasma carotenoids, tocopherols, ascorbic acid, uric acid and characteristics of study participants are presented in Table 1. The plasma total carotenoid (lutein+β-carotene+lycopene) concentrations were significantly increased within 15 days of supplementation of lutein (12 mg/d, p<0.05), β-carotene (12 mg/d, p<0.01), lycopene (12 mg/d, p<0.01) or mixed carotenoids (4 mg/d each of lutein, β-carotene, lycopene, p<0.01), and maintained those levels throughout the study period as shown in FIG. 2. The plasma total carotenoid levels of all carotenoid supplemented groups were significantly higher than those of the placebo group (P<0.05) at d 15, 29, 43 and 57. The plasma lutein concentrations were significantly increased (p<0.005) on day 15 in lutein group and mixed carotenoid group so that the values were 514% and 228% of the baseline in lutein and mixed carotenoid groups respectively. Those levels were maintained throughout the study period (FIG. 3). There was no increase in plasma lutein levels in placebo, β-carotene, and lycopene groups during the intervention period. The concentrations of plasma β-carotene were significantly increased within 15 days in β-carotene group (p<0.01) and mixed carotenoid group (p<0.05) so that the levels were reached to 387% and 146% in β-carotene and mixed carotenoid groups respectively. Placebo, lutein, and lycopene supplemented groups did not show any increase in plasma β-carotene levels (FIG. 4). The plasma lycopene concentrations were significantly increased in lycopene group within 15 days (p<0.05), whereas placebo, lutein, and β-carotene groups showed significantly lower levels of plasma lycopene concentrations during the intervention period as shown in FIG. 5. The plasma lycopene concentrations of mixed carotenoid group were between 110%-125% of baseline during the intervention period.

Plasma lutein, β-carotene and lycopene concentrations were significantly and selectively increased to 0.90, 1.47 & 1.07 μM respectively within 15 days of 12 mg each of lutein, β-carotene or lycopene supplementation whereas other carotenoid levels were maintained or lower than baseline levels. These increased carotenoid concentrations were much higher than those of 90% of National Health and Nutrition Examination Survey (NHANESIII) plasma carotenoid levels (lutein, 0.67; β-carotene, 0.91; lycopene, 0.70 μM) in the same age, gender and ethnic group (50-70 yr, non Hispanic white women, n=1017) as our study participants. However, plasma carotenoid levels in mixed carotenoid group, who received 4 mg each of lutein, β-carotene and lycopene, reached 0.40, 0.50 & 0.52 μM for lutein, β-carotene and lycopene respectively on day 15, which are within the levels of median to seventy-five percentile of NHANES III same age, gender and ethnic group.

TABLE 1
Anthropometric characteristics of study participants

| | Placebo | Mixed Car | Lutein | β-carotene | Lycopene |
|---|---|---|---|---|---|
| Group | (n = 6) | (n = 8) | (n = 8) | (n = 7) | (n = 8) |
| Age (yrs) | 64 ± 5 | 59 ± 6 | 62 ± 5 | 59 ± 5 | 56 ± 6 |
| Height (cm) | 161.5 ± 4.6 | 166.9 ± 6.2 | 164.1 ± 5.4 | 168.0 ± 5.0 | 166.4 ± 8.6 |
| Weight (kg) | 65.8 ± 6.2 | 73.0 ± 9.2 | 69.7 ± 8.0 | 73.9 ± 15.1 | 66.3 ± 9.8 |
| BMI | 25.2 ± 1.4 | 26.2 ± 3.2 | 25.9 ± 2.4 | 26.1 ± 4.5 | 24.0 ± 3.1 |

Values are means ± SD

Plasma lutein response to the mixed carotenoid supplementation (4 mg/d each of lutein, β-carotene and lycopene) was significantly correlated (r=0.804, p=0.016) with the baseline concentration of lutein (data not shown). When the study participant had the higher baseline plasma lutein concentration, the increase of plasma lutein level in response to the mixed carotenoid was the greater. The plasma β-carotene response to the mixed carotenoid supplementation also tends to be correlated with the baseline concentration of β-carotene (r=0.677, p=0.065). However, plasma lycopene response to the mixed carotenoid supplementation was not as well correlated.

The effects of carotenoid supplementations on DNA damage are shown in Table 2. The basal DNA damage levels were significantly higher in the β-carotene and lycopene groups as compared to that of placebo group (p<0.05). The basal DNA damage levels were significantly decreased as early as d 15 in mixed carotenoid p<0.01), β-carotene (p<0.01) and lycopene (p<0.05) groups as compared to those of day 1. The placebo group did not show any significant change in basal DNA damage during the intervention period.

TABLE 2
The effects of carotenoid supplementation against basal lymphocyte DNA
damage (%, Means ± SD)

| Group | Day 01 | Day 15 | Day 29 | Day 43 | Day 57 |
|---|---|---|---|---|---|
| Basal DNA damage | | | | | |
| Placebo | 8.7 ± 2.0 | 9.0 ± 2.5 | 10.6 ± 3.2 | 9.2 ± 4.1 | 9.9 ± 3.8 |
| Mixed Carotenoids | 10.9 ± 1.5 | 8.6 ± 1.6^{b} | 7.9 ± 1.8^{a} | 7.1 ± 1.4^{b} | 7.0 ± 1.3*^{b} |
| Lutein | 10.6 ± 1.4 | 9.4 ± 2.1 | 9.5 ± 1.4 | 7.7 ± 1.5^{b} | 7.1 ± 1.7*^{c} |
| β-carotene | 12.4 ± 2.7** | 9.7 ± 2.4^{b} | 8.6 ± 2.9^{a} | 9.4 ± 2.4^{a} | 8.0 ± 1.8^{b} |
| Lycopene | 11.9 ± 2.6** | 10.0 ± 3.5^{a} | 9.0 ± 2.6^{a} | 7.5 ± 1.9^{b} | 6.8 ± 1.6*^{c} |

Note:
Compare to placebo group, * P < 0.05, ** P < 0.01, *** P < 0.001,
Compare to Day 1, a P < 0.05, b P < 0.01, c P < 0.001

When DNA was challenged with hydrogen peroxide (lymphocytes were treated with H₂O₂ at 10 micromolar for 10 min), DNA susceptibility against oxidative damage were significantly improved by mixed carotenoid, β-carotene and lycopene supplementation (p<0.05) at d 57 (Table 3). Placebo and lutein groups did not show any significant change during the intervention. The results indicate that carotenoid supplementation can effectively protect against lymphocyte DNA damage and that the protective effect of mixed carotenoid supplementation against DNA damage is rapid and consistent. In addition, the protective effect of the mixed carotenoid supplementation increased over time which indicates that the mixed carotenoid supplementation has a cumulative positive effect on the subjects.

TABLE 3
The effects of carotenoid supplementation against hydrogen peroxide induced
lymphocyte DNA damage. (%, Means ± SD)

| | | | | | |
|---|---|---|---|---|---|
| Group | Day 01 | Day 15 | Day 29 | Day 43 | Day 57 |

Resistance of DNA against oxidative stress¹

| | | | | | |
|---|---|---|---|---|---|
| Placebo | 42.1 ± 5.4 | 44.6 ± 6.0 | 39.7 ± 2.8 | 43.0 ± 7.1 | 40.6 ± 7.6 |
| Mixed Carotenoids | 44.2 ± 7.0 | 43.2 ± 9.2 | 42.6 ± 7.7 | 37.1 ± 11.3 | 36.4 ± 6.2^{a} |
| Lutein | 42.8 ± 6.8 | 43.5 ± 6.2 | 43.1 ± 5.6 | 41.5 ± 9.3 | 39.8 ± 8.4^{a} |
| β-carotene | 48.2 ± 6.1 | 44.5 ± 8.9 | 41.1 ± 6.2 | 44.2 ± 5.7 | 38.0 ± 4.8 |
| Lycopene | 50.5 ± 8.9* | 49.2 ± 10.1 | 51.1 ± 3.5** | 50.0 ± 6.5 | 42.5 ± 6.5^{b} |

Note:
Compare to placebo group, * P < 0.05, ** P < 0.01, *** P < 0.001,
Compare to Day 1, a P < 0.05, b P < 0.01, c P < 0.001
¹DNA was challenged with 10 μM of H₂O₂ for 10 min

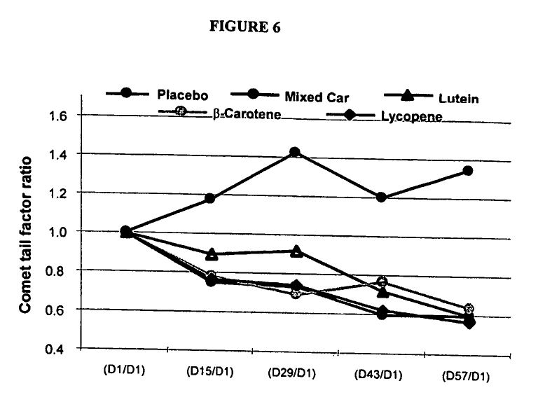

FIG. 6 shows the percent of comet tail ratio that is each day value was divided by the value of day 1. DNA damage was increased in the placebo group whereas basal DNA damage was significantly decreased in mixed carotenoid, lutein, β-carotene, lutein and lycopene groups and these values were significantly different from placebo group at d 57 (p<0.005). The study shows that there was a significant decrease in basal DNA damage after supplementing 12 mg of single or combination of carotenoids in elderly women for 15 days and the protective effect was maintained throughout the study period for 57 days in women (50-70 yr).

The results indicate that carotenoid supplementation can effectively protect against lymphocyte DNA damage and that the protective effect of mixed carotenoid supplementation against DNA damage is rapid and consistent. In addition, the protective effect of the physiologic dose of mixed carotenoid supplementation increased over time, which indicates that the mixed carotenoid supplementation has a cumulative positive effect on the subjects. Therefore, the study confirms that oral administration of the composition of the present invention is effective as a nutritional supplement, either therapeutically or prophylactically, for example, in preventing the severity or delaying or preventing the onset of a disease.

While the present invention has been described in terms of specific methods and compositions, it is understood that variations and modifications will occur to those skilled in the art upon consideration of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

1. A pharmaceutical composition for use in decreasing DNA damage comprising an effective daily dose of about 0.1 to 20 mg lutein, and at least one of the group consisting of beta-carotene and lycopene in amounts sufficient to act synergistically with lutein.

2. The pharmaceutical composition of claim 1, wherein the composition further comprises at least one of about 0.1mg to 20 mg beta-carotene or about 0.1 to 20 mg lycopene.

3. The pharmaceutical composition of claim 1, wherein the composition further comprises a lipophilic component.

4. The pharmaceutical composition of claim 1, wherein the composition further comprises a carotenoid-containing dry powder in the form of a multicore structure in which at least two cores of a multicore structure comprise one or more different carotenoids of the group consisting of substantially purified lutein, beta-carotene, and lycopene.

5. The pharmaceutical composition of claim 4, wherein the carotenoid-containing dry powder is formed into at least one of drink preparations, tablets, sugar coated tablets, hard gelatin capsules, soft gelatin capsules, and cellulose capsules.

6. A nutritional composition suitable for use in protecting against a free radical associated disorder, comprising a daily dose of at least two carotenoids selected from the group consisting of substantially purified lutein, beta-carotene, and lycopene.

7. The nutritional composition of claim 6, wherein the daily dose of at least two carotenoids is selected from about 0.1% to 50% by weight beta-carotene, about 0.1% to 50% by weight lycopene, and about 0.1% to 50% by weight lutein.

8. A method of decreasing oxidative damage in a subject comprising: administering a synergistic combination of carotenoids to the subject, wherein the synergistic combination comprises at least two carotenoids selected from the group consisting of lutein, beta-carotene, and lycopene.

9. The method of claim 8, wherein the synergistic combination of carotenoids is selected from the group consisting of a daily unit dose of about 0.1 mg to 20 mg beta-carotene, about 0.1 to 20 mg lycopene, and about 0.1 to 20 mg lutein to the subject.

10. The method of claim 8, wherein the method comprises administering about 0.5 mg to 10 mg beta-carotene, about 0.5 to 10 mg lycopene, and about 0.5 to 10 mg lutein to the subject.

11. The method of claim 8, wherein the synergistic combination of carotenoids is selected from the group consisting of a daily unit dose of about 1 part of beta-carotene, 0.02 to 20 parts of lycopene and 0.02 to 20 parts of lutein.

12. The method of claim 8, wherein the synergistic combination of carotenoids is selected from the group consisting of a daily unit dose of about 1 part of beta-carotene, 0.1 to 2 parts of lycopene and 0.1 to 2 parts of lutein.

13. The method of claim 8, wherein the method further comprises administering a lipophilic component, such that antioxidant capacity in the aqueous and lipid compartments of plasma is increased.

14. The method of claim 8, wherein the method further comprises administering a carotenoid-containing dry powder in the form of a multicore structure in which at least two cores of a multicore structure comprise one or more different carotenoids of the group consisting of substantially purified lutein, beta-carotene, and lycopene.

15. The method of claim 14, wherein the method comprises administering the carotenoid-containing dry powder in a form selected from the group consisting of drink preparations, tablets, sugar coated tablets, hard gelatin capsules, soft gelatin capsules, and cellulose capsules.

16. A method of reducing effects of aging in a subject comprising: administering a synergistic combination of carotenoids to the subject, wherein the synergistic combination comprises at least two of the group consisting of lutein, beta-carotene, and lycopene, whereby DNA damage in the subject is decreased thereby reducing the effects of aging.

17. The method of claim 16, wherein the synergistic combination of carotenoids is selected from the group consisting of a daily unit dose of about 0.1 mg to 20 mg beta-carotene, about 0.1 to 20 mg lycopene, and about 0.1 to 20 mg lutein to the subject.

18. The method of claim 16, wherein the method comprises administering about 0.5 mg to 10 mg beta-carotene, about 0.5 to 10 mg lycopene, and about 0.5 to 10 mg lutein to the subject.

19. The method of claim 16, wherein the method comprises a carotenoid-containing dry powder in the form of a multicore structure in which at least two cores of a multicore structure comprise one or more different carotenoids of the group consisting of substantially purified lutein, beta-carotene, and lycopene.

20. The method of claim 19, wherein the method comprises administering the carotenoid-containing dry powder in a form selected from the group consisting of drink preparations, tablets, sugar coated tablets, hard gelatin capsules, soft gelatin capsules, and cellulose capsules.
